(19) United States
(12) Patent Application Publication
(10) Pub. No.: US 2018/0140630 A1
(43) Pub. Date: May 24, 2018

(54) COMBINATION OF AN H2-RECEPTOR ANTAGONIST, ANTACID, AND ALGINIC ACID TO TREAT EPISODIC HEARTBURN

(71) Applicants: M. Michael Wolfe, Beachwood, OH (US); Steven B. Landau, Wellesley, MA (US); Abhijeet Waghray, Westlake, OH (US); Nisheet Waghray, Westlake, OH (US)

(72) Inventors: M. Michael Wolfe, Beachwood, OH (US); Steven B. Landau, Wellesley, MA (US); Abhijeet Waghray, Westlake, OH (US); Nisheet Waghray, Westlake, OH (US)

(21) Appl. No.: 15/820,821

(22) Filed: Nov. 22, 2017

Related U.S. Application Data
(60) Provisional application No. 62/425,866, filed on Nov. 23, 2016.

(51) Int. Cl. A61K 33/08; A61K 9/00; A61P 1/04; A61K 31/734; A61K 31/426; A61K 31/4164; A61K 31/341

(52) U.S. Cl. CPC A61K 33/08; A61K 9/0053; A61P 1/04; A61K 31/341; A61K 31/426; A61K 31/4164; A61K 31/734

(57) ABSTRACT

A combination of an antacid, alginate, and a histamine H₂-receptor antagonist, and methods of using the same for providing fast and lasting relief of symptoms of episodic heartburn are provided.

### CROSS-REFERENCE TO RELATED APPLICATION

This application claims priority to U.S. Provisional Application No. 62/425,866, filed Nov. 23, 2016, the disclosure of which is incorporated herein by reference in its entirety.

### FIELD OF THE INVENTION

The invention relates to a combination of alginate with an H₂-receptor antagonist and an antacid to treat episodic heartburn.

### BACKGROUND OF THE INVENTION

Gastroesophageal reflux disease (GERD) is principally a motility disorder, and its most common manifestation is heartburn, which is defined as the sensation of burning beneath the sternum (1). While most patients are not hypersecretors of gastric acid, the therapy of GERD has historically generally not been directed at the underlying pathophysiology (1, 2). Rather, therapy is aimed at rendering the refluxate less toxic (i.e. less acidic) to the esophageal mucosa, tilting the balance between offensive and defensive forces toward the side of mucosal protection. Despite the large number of potential lifestyle modifications available, patients typically require pharmacological therapy to control symptoms, and although these measures should be discussed with patients, the use of medication should not be delayed except in cases of very mild and infrequent heartburn.

Approximately 7-10 percent of all people report heartburn daily, and about 25-40 percent monthly report episodic heartburn (1). Episodic heartburn is often associated with the ingestion of different foods, and it is also referred to as “sour stomach” or “indigestion.” Although different foods, such as coffee, mints, fatty foods, alcohol, and chocolate, are usually implicated in the etiology of episodic heartburn, these symptoms can be caused by any type of food in certain people. Moreover, in many people, there is no inciting agent that can be identified, rather the disorder occurs without any known provocation.

Proton pump inhibitors (PPIs) are widely used class of agents employed in the treatment of GERD. They act on the final common pathway of acid secretion, the H+/K+ ATPase enzyme located on the apical membrane of the gastric parietal cell.

PPIs have several drawbacks in the context of episodic heartburn. All PPIs are prodrugs and bind only to activated proton pumps (1, 2). Thus, the appropriate time to administer a PPI is before a meal to ensure that drug is circulating during the period of parietal cell activation. Maximal pump activation occurs with the first meal after an overnight fast, making breakfast the best time to take a PPI. In addition, PPI therapy for GERD is usually continuous, with either daily or twice daily dosing. Moreover, relapse rates after discontinuation of therapy are approximately 75%, confirming the need for long-term maintenance therapy. For many patients with only episodic heartburn, taking a PPI before a meal or on a long-term basis would be unnecessary.

Studies have examined the possibility of intermittent therapy with PPIs, also known as “on-demand” therapy, in patients with nonerosive reflux disease or mild reflux esophagitis. Early studies of “weekend PPI therapy” given three days per week were disappointing, with symptom relief at one year significantly lower with weekend therapy compared with daily dosing. Finally, Barrison et al. (3) reported that approximately 70% of primary care physicians prescribed PPIs before bedtime or without instruction, and more recently Sheikh et al. (4) found that consumers were more likely to be suboptimal users compared to physicians who prescribed PPIs. In the latter study, suboptimal use of PPIs was associated with inadequate symptom control (4). For this reason and because of their potency and the need to use these agents continuously, they are not appropriate for use in patients with episodic heartburn.

Antacids are a class of agents that directly neutralize gastric acid. They are among the earliest medications used for gastrointestinal disorders, with ground coral powder (calcium carbonate) described as a remedy for dyspepsia in ancient Rome by Pliny The Elder (5). Current antacid preparations include magnesium or aluminum hydroxides and calcium carbonate. Powdered sodium bicarbonate is also available, but it is less frequently used. These agents provide rapid but short-lived relief of heartburn (30-60 minutes), necessitating frequent dosing. Currently, antacid therapy is useful in the management of mild, uncomplicated GERD in patients with infrequent symptoms (6). While antacids effectively neutralize acid and can provide temporary symptomatic relief, they have several disadvantages. If used alone to block episodic heartburn, frequent dosing is required and are not substantially effective to neutralize nocturnal acid. Although antacids are a very safe class of drug, they can alter bowel function and may be occasional adverse effects associated with their use, including diarrhea (with magnesium-containing formulations) or constipation (with aluminum-based formulations).

Alginic acid is an excipient that by itself is not effective in alleviating heartburn. Some small studies have demonstrated that antacid/alginic acid combinations are superior to placebo for the relief of heartburn, while others attribute the effectiveness of the combination to the antacid alone (7, 8). For these reasons, alginic acid is regarded as an excipient in such formulations.

Histamine Hz-receptor antagonists (H2RAs) inhibit acid secretion by binding to the histamine-2 receptor on the basolateral membrane of the parietal cell. Histamine, secreted by the gastric enterochromaffin-like cell, is the principal stimulus for gastric acid secretion, and the inhibition of this paracrine effect effectively decreases acid production (1). Early clinical studies demonstrated little benefit of H2RAs in the treatment of GERD, which may have been due, however, to the fact that the dose of H2RA required to effectively treat duodenal ulcer is insufficient to treat GERD. Later studies using appropriate H2RA dosing have consistently shown a clinical effect greater than placebo, both in the control of reflux symptoms and the healing of erosive esophagitis. The available H2RAs (cimetidine, ranitidine, famotidine, and nizatidine) appear to be equally effective in treating GERD.

The effectiveness of H2RA therapy depends upon the severity of erosive disease, with response rates of nearly 80% in Grade I-II esophagitis, but response rates of only 30-50% in Grade III-IV disease (2, 9). Thus, the use of H2RAs has been relegated mostly to the management of mild heartburn, often using over-the-counter (OTC) preparations (10).

While H2RAs and antacids are widely used, they are not optimal drugs for treating episodic heartburn. The problem with antacids is that, while working within seconds to minutes to chemically neutralize refluxed acid in the esophagus, they provide only transient relief. This disadvantage is more apparent in many people when heartburn occurs at night, during which sustained symptomatic relief is not provided. The problem with histamine H2RAs is that relief is typically not experienced until about 45 minutes to about two hours after the medication is ingested.

In U.S. Pat. No. 5,229,137, incorporated by reference herein in its entirety, compositions with both antacids and H2Ras are described, along with methods of administering such compositions to human patients to relieve gastric distress. In the past, the simultaneous administration of antacids and H2RAs had been discouraged based upon studies demonstrating that antacids decreased the absorption and subsequent blood levels of the latter class of drugs, thereby decreasing their ability to inhibit acid secretion (11). However, the combination of antacids and H2RAs was subsequently shown to provide superior inhibition of intragastric acidity than either agent alone (12). Moreover, the combination has proven to be superior to either agent alone for providing immediate, temporary, and sustained relief to people who suffer from episodic heartburn.

Nevertheless, despite their benefit, such formulations, including Pepcid Complete® (a combination of famotidine hydrochloride and calcium carbonate), are not universally beneficial in treating patients suffering from episodic heartburn.

There remains a need for formulations that can treat patients suffering from episodic heartburn that do not respond satisfactorily to a combination of a histamine H₂-receptor antagonist and an antacid.

### SUMMARY OF THE DISCLOSURE

Provided herein are improved compositions, formulations and methods of treating episodic heartburn.

An oral pharmaceutical medication is described that provides immediate and sustained relief from pain, discomfort and/or symptoms associated with acid reflux, episodic heartburn, or both, in a human. The oral pharmaceutical medication comprises an antacid in an amount effective to substantially neutralize gastric acid, alginate, and an H2RA in an amount effective to substantially inhibit or block gastric acid secretion.

A method is described that provides immediate and sustained relief from pain, discomfort and/or symptoms associated with episodic heartburn. Also a method is described that provides immediate and sustained relief from pain, discomfort and/or symptoms associated with acid reflux. An antacid in an amount effective to substantially neutralize gastric acid, alginate, and an H2RA in an amount effective to substantially inhibit or block gastric acid secretion are administered to a human, together or substantially together. The method provides the human with immediate and sustained relief from pain, discomfort and/or symptoms associated with acid reflux, episodic heartburn, or both.

### BRIEF DESCRIPTION OF THE DRAWINGS

FIGS. 1A and 1B illustrate a questionnaire used to assess gastrointestinal reflux disease symptoms. FIG. 1A lists questions about the seven symptoms and FIG. 1B lists questions about eight symptoms.

FIGS. 2 and 3 show a study design protocol in which patients are divided into three groups that are tested with each of (i) Pepcid Complete® and alginate, (ii) alginate and a placebo, and (iii) Pepcid Complete® and a placebo.

### DETAILED DESCRIPTION

One aspect is an oral pharmaceutical medication for providing immediate and sustained relief from pain, discomfort and/or symptoms associated with acid reflux, episodic heartburn, or both, in a human. The oral pharmaceutical medication comprises an antacid in an amount effective to substantially neutralize gastric acid, alginate, and a histamine H₂-receptor antagonist in an amount effective to substantially inhibit or block gastric acid secretion.

In various embodiments, the amounts of each of the gastric acid, alginate, and an histamine H₂-receptor antagonist (H2RA) are effective to provide immediate and sustained relief from pain, discomfort and/or symptoms associated with episodic heartburn.

In various embodiments, the oral pharmaceutical medication is effective to provide immediate and sustained relief from pain, discomfort and/or symptoms associated with episodic heartburn.

The terms “immediate relief” and “sustained relief” are referred to in a broad sense herein. Particularly, “immediate relief” means that relief obtained from pain, discomfort and/or symptoms associated with episodic heartburn occurs within about 5-10 minutes following ingestion of the active ingredients or an antacid. “Sustained relief” refers to relief obtained from pain, discomfort and/or symptoms associated with episodic heartburn which lasts in duration for over about 4-6 hours following ingestion of the active ingredients or the H2RA. “Immediate and sustained relief” includes immediate, temporary and sustained relief which starts within about 5-10 minutes following ingestion of the active ingredients that continues and remains constant for at least about 4-6 hours after ingestion of the oral pharmaceutical medication.

The immediate and sustained relief of the oral pharmaceutical medication may last longer than when the human is orally treated with either the antacid, the alginate, or the antacid and the alginate. The immediate sustained relief may be faster than, and last at least about as long as when the human is orally treated with either the histamine H₂-receptor antagonist or the H2RA and the antacid.

In some embodiments, the oral pharmaceutical medication is effective to provide temporary relief from pain, discomfort and/or symptoms associated with episodic heartburn in the human. “Temporary relief” refers to relief from pain, discomfort and/or symptoms associated with episodic heartburn which lasts in duration on the order of between about 30 minutes and 90 minutes after ingestion of the active ingredients or an antacid.

The term “episodic heartburn” herein refers to the sensation of burning under the sternum (breastbone) usually, but not necessarily, associated with the ingestion of different foods. Also included in this definition of “episodic heartburn” is sour stomach, indigestion and waterbrash/regurgitation. Symptoms of episodic heartburn may further include one or more of (a) burning pain in the chest, (b) sensation of pressure or discomfort inside the chest, (c) food coming back to the mouth, (d) acid in the mouth, (e) a sour taste in the mouth, (f) frequent gurgling in the stomach or abdomen, (g) a feeling of pressure in the throat, (h) sensation of a lump in the throat, (i) nausea, (j) urge to vomit, (k) burning pain in the throat, (j) bloating, (k) belching, (l) flatulence, (m) feeling full only after eating a little, (n) bad breath, (o) coughing, and (p) hoarseness. The oral formulation may relieve one or more of these symptoms. Further, the frequency and severity of these symptoms over a period of time, such as a week, can be assessed.

The term “acid reflux” refers to regurgitation of gastric fluid into the esophagous. As with heartburn, symptoms of acid reflux may further include one or more of (a) burning pain in the chest, (b) sensation of pressure or discomfort inside the chest, (c) food coming back to the mouth, (d) acid in the mouth, (e) a sour taste in the mouth, (f) frequent gurgling in the stomach or abdomen, (g) a feeling of pressure in the throat, (h) sensation of a lump in the throat, (i) nausea, (j) urge to vomit, (k) burning pain in the throat, (j) bloating, (k) belching, (l) flatulence, (m) feeling full only after eating a little, (n) bad breath, (o) coughing, and (p) hoarseness. The oral formulation may relieve one or more of these symptoms. Further, the frequency and severity of these symptoms over a period of time, such as a week, can be assessed.

The term “antacid(s),” is used broadly herein and refers to those agents which can block gastric acid and/or bile salts by neutralization, and/or inhibit the proteolytic activity of pepsin. The antacid may be a conventional antacid that is well known and widely used in the treatment of a variety of excess acid-related gastrointestional dysfunctions including acid indigestion, heartburn, sour stomach and ulcers. The antacid may include one or more of aluminum hydroxides, calcium carbonates, magnesium hydroxides, sodium bicarbonates, magnesium carbonates, magnesium oxides and the like, as well as those antacids that are commercially available. The antacid may be a flavored antacid. The antacid may be a high potency antacid. Antacids may be used in dosage amounts conventionally used for treatment of a variety of excess acid-related gastrointestional dysfunctions, as discussed above.

Various amounts of antacids may be used. The amount of total antacid may be from 500 to 1500 mg, 600 to 1400 mg, 700 to 1300 mg, 800 to 1200 mg, about 1000 mg, or 1000 mg. Typical dosages include about 30 mls or 2 tablespoons of a high-potency antacid having an acid-neutralizing capacity equal to the present formulations of, for example, Maalox Plus®, Mylanta-II®. Calcium carbonate may be used as an antacid. The amount of calcium carbonate may be from 400 to 1200 mg, 500 to 1100 mg, 600 to 1000 mg, 700 to 900 mg, about 800 mg, or 800 mg. Magnesium hydroxide may be used as an antacid. The amount of magnesium hydroxide may be from 100 to 200 mg, 120 to 180 mg, 140 to 175 mg, about 165 mg, or 165 mg.

Alginate, also known as alginic acid and commonly in the form of sodium alginate, are polymers found in at least in the cell walls of brown seaweeds. Alginate may be refined from brown seaweeds, such as those of the phylum Phaeophyceae. The refined alginate can be in the form of a sodium salt, i.e. sodium alginate. Alginates can form viscous solutions and gels. Such viscous solutions and gels can form in the stomach as well. In the compositions and methods described herein, the alginate may be a metallic salt or in the form of alginic acid. Also, the alginate used in the oral pharmaceutical medication may be sodium alginate.

Various amounts of alginate may be used in the oral pharmaceutical medication. About 100 mg, 200 mg, 300 mg, 400 mg, 500 mg, 600 mg, 700 mg, 800 mg, 900 mg, 1000 mg, 1100 mg, 1200 mg, 1300 mg, 1400 mg and 1500 mg may be used. The amount of alginate may be from 100 to 1500 mg, 200 to 1300 mg, 400 to 1200 mg, 500 to 1100 mg, 600 to 1100 mg, 700 to 1100 mg, 800 to 1000 mg, or 900 to 1100 mg. More particularly, about 1000 mg of sodium alginate may be used.

After being administered in a dosage form for a gastric disease, such as acid reflux, alginic acid is thought to create a viscous layer atop the gastric acid pool. The viscous layer is thought to decrease acid reflux by physically preventing acid from entering the esophagus and by delivering co-administered antacids to the esophagus.

The term “histamine H₂-receptor antagonist(s)” or “H2RA” is referred to herein in a broad sense and is meant to include those agents that inhibit or block the secretion of gastric acid by binding to a specific histamine receptor on the parietal (acid-secreting) cell membrane located in the stomach. The histamine H₂-receptor antagonist may be one or more of cimetidine, ranitidine, nizatidine and famotidine.

Various amounts of H2RAs may be used in the oral pharmaceutical medication.

In some embodiments, the H2RA is cimetidine. The amount of cimetidine may be from 50 to 400 mg, 75 to 450 mg, 100 to 500 mg, 200 to 600 mg, 300 to 450 mg, about 400 mg, or 400 mg. Alternatively, cimetidine may be present in an amount of between about 200 mg and about 300 mg.

In some embodiments, the H2RA is ranitidine. The amount of ranitidine may be from 25 to 300 mg, 50 to 200 mg, 75 to 175 mg, 100 to 175 mg, about 150 mg, or 150 mg. Alternatively, ranitidine may be present in an amount of between about 100 mg and about 150 mg.

In some embodiments, the H2RA is nizatidine. The amount of nizatidine may be from 25 to 300 mg, 50 to 200 mg, 75 to 175 mg, 100 to 175 mg, about 150 mg, or 150 mg. Alternatively, nizatidine may be present in an amount of between about 100 mg and about 150 mg.

In some embodiments, the H2RA is famotidine. The amount of famotidine may be from 2 to 30 mg, 5 to 20 mg, 7 to 12 mg, 17 to 22 mg, 8 mg to 12 mg, 18 mg to 21 mg, 9 mg to 11 mg, 19 mg to 21 mg, about 20 mg, about 10 mg, or 10 mg. In some embodiments, the daily dose of famotidine is from 5 mg to 40 mg.

In some embodiments, additional components may be included to slow release of histamine H₂-receptor antagonist(s), or to otherwise prolong the time period over which H2RA(s) are released in active in the stomach. If the oral pharmaceutical medication is a tablet, then such components can be polymers that are coated on a tablet or a portion, or layer, of the tablet that is enriched for H2RA(s). In other words, the H2RA(s) in the oral pharmaceutical medication can be in an extended release form.

In some embodiments, the dosage form is a rapid melt formulation. The rapid melt formulation dissolves in less than one minute when placed on the tongue without the need to drink water or chew. One or more of the components in the rapid melt formulation may be microencapsulated to avoid unpleasant taste. The rapid melt formulation may be formulated for buccal administration.

Including alginic acid or alginate with an antacid and an H2RA provides greater symptom relief over formulations of the prior art. Even if reduced regurgitation from sodium alginate could be predicted due the formation of a barrier against the reflux of gastric contents, it is surprising and unexpected that alginate contributes to superior relief of episodic heartburn and overall clinical improvement, as assessed using a universally-accepted global symptom score. The combination of famotidine, antacid, and alginate provides superior relief of episodic heartburn, as compared to either a corresponding mixture of famotidine and antacid, without the alginate, or to alginate alone. Relief of episodic heartburn is measured in terms of symptom, frequency, and severity scores.

It has not been expected that adding alginate to a combination of H2RA(s) and antacid would provide for superior relief of episodic heartburn. Even if alginates mechanically block reflux of gastric acid into the esophagus, like antacids they have a very short duration of action such that frequent dosing is needed. See paragraph [0008] of U.S. Patent Application No. 2013/0017263.

In some embodiments, the oral pharmaceutical medication includes a combination of famotidine, sodium alginate, and antacid. In some embodiments, the amount of famotidine is from 9 mg to 21 mg, about 10 mg, or about 20 mg. In some embodiments, the amount of alginate is from 900 to 1100 mg, or about 1000 mg. The antacid, as defined above, is in an amount between 800 to 1200 mg.

In some embodiments, the oral pharmaceutical medication includes a combination of famotidine, sodium alginate, calcium carbonate and magnesium hydroxide. The amount of famotidine may be from 9 mg to 21 mg, about 10 mg, or about 20 mg. The amount of alginate may be from 900 to 1100 mg, or about 1000 mg. The amount of calcium carbonate may be from 700 to 900 mg, or about 800 mg. The amount of magnesium hydroxide may be from 150 to 175 mg, or about 165 mg.

For example, a typical dosage amount for providing immediate and sustained relief from episodic heartburn in an adult is about 30 ml of a high potency flavored antacid, or the equivalent thereof, and about 200 mg to about 300 mg of cimetidine, or 100 mg to about 150 mg of ranitidine, administered between about one and about four times per day. The oral pharmaceutical medication can be taken on an as-needed basis, such as whenever pain or symptoms associated with episodic heartburn, acid reflux, or both, is experienced. The oral pharmaceutical medication can be taken in advance of drinking coffee and other caffeinated beverages, or eating foods that are known to provoke episodic heartburn, such as spicy and/or high fat foods, peppermint, and those containing coffee or caffeine.

The oral pharmaceutical medications can be conveniently prepared from, for example, commercially available antacids and H2RA, and may be formulated into liquid, solid, or rapid melt dosage forms or combinations thereof. For example, the pharmaceutical medications may be taken as a single unitary dose containing the antacid, alginate, and the H2RA in a liquid or solid dosage form. The ingredients may be taken substantially together. By the term “substantially together,” it is meant herein that when the active ingredients are taken in separate dosage forms, they can be consumed either simultaneously or within a period of time such that the immediate, temporary and sustained relief obtained is constant and uninterrupted. For example, the active ingredients may be taken together or within a few seconds to a few minutes of one another. A single unitary dose which includes all active ingredients in liquid form may be taken.

Alternatively, the ingredients may be taken separately in the same or different dosage forms, such as taking the antacid and alginate as a liquid dose and the H2RA as a solid dose or vice versa, or taking them separately as either solid or liquid doses. The H2RA can be in a solid dose with additional polymers and components providing for time release of H2RA. In some embodiments, the dosage form is a rapid melt formulation that dissolves in less than one minute when placed on the tongue without the need to drink water or chew. One or more of the components in the rapid melt formulation may be microencapsulated to avoid unpleasant taste. The rapid melt formulation may be formulated for buccal administration.

In some embodiments, the oral pharmaceutical medication further comprises a pharmaceutically acceptable carrier.

The oral pharmaceutical medication may be in a liquid dosage form or a solid dosage form.

In some embodiments, the oral pharmaceutical medication further includes an effective amount of an antiflatulent. Antiflatulents that may be used include those which are conventionally used in the treatment of gastrointestinal dysfunction, such as, for example, simethicone. Antiflatulents may be used in the present invention in dosage amounts conventionally used in the treatment of gastrointestinal dysfunction.

The pharmaceutical compositions may be in a form suitable for oral use, for example, as tablets, lozenges, aqueous or oily suspensions, dispersible powders or granules, emulsions, hard or soft capsules, syrups or elixirs. Compositions intended for oral use may be prepared according to any method known to the art for the manufacturer of pharmaceutical compositions and such compositions may contain one or more agents such as, for example, sweetening agents, flavoring agents, coloring agents and the like, in order to provide a pharmaceutically elegant and palatable preparation. Tablets contain the active ingredient in admixture with nontoxic pharmaceutically acceptable excipients which are suitable for manufacture of tablets. These excipients may be, inert diluents, for example, calcium carbonate, sodium carbonate, lactose, calcium phosphate or sodium phosphate; granulating and disintegrating agents, for example, or maize starch; binding agents, for example, starch, gelatine or acacia, and lubricating agents, for example, magnesium stearate or stearic acid. The tablets may be uncoated or they may be coated by known techniques to delay disintegration and absorption in the gastrointestinal tract and thereby provide an even longer sustained action over a period of time.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with a suitable oil medium, for example, arachis oil, liquid paraffin or olive oil.

Aqueous suspensions contain the active ingredients in admixture with excipients suitable for the manufacture of aqueous suspensions. Such excipients may be suitable suspending agents, for example, sodium carboxymethyl cellulose, methyl cellulose, hydroxy propyl methyl cellulose, polyvinylpyrrolidone, gum tragacanth and gum acacia; dispersing or wetting agents may be any suitable naturally occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol, for example, polyoxyethylene sorbitol monnoleate, or condensation product of ethylene oxide with partial esters derived from fatty acids and hexitol and anhydrides, for example, polyoxyethelyne sobirtan monooleate. The aqueous suspensions may also contain one or more suitable preservatives, for example, ethyl, or n-propyl, p-hydroxy benzoate, one or more suitable coloring agents, one or more suitable flavoring agents and one or more suitable sweetening agents, such as sucrose, saccharin, sucralose, or aspartame.

Dispersible powders and granules suitable for preparation of an aqueous suspension by the addition of water provide the active ingredient in admixture with a dispersing or wetting agent, suspending agent and on or more preservatives. Suitable dispersing or wetting agents and suspending agents may be exemplified by those already mentioned above. Additional suitable excipients, for example, sweetening, flavoring and coloring agents, may also be present.

Syrups and elixirs may be formulated with suitable sweetening agents, for example, glycerol, sorbitol, or sucrose. Such formulations may also contain suitable demulcents, preservatives and flavoring and coloring agents.

Another aspect is a method of providing immediate and sustained relief from pain, discomfort and/or symptoms associated with episodic heartburn, acid reflux, or both. In the method, an antacid in an amount effective to substantially neutralize gastric acid, alginate, and an H2RA in an amount effective to substantially inhibit or block gastric acid secretion are, together or substantially together, orally administered to a human. The method provides immediate and sustained relief from pain, discomfort and/or symptoms associated with episodic heartburn, acid reflux, or both.

In various embodiments, any of the oral pharmaceutical medications described above in the “Detailed Description” are administered to the human. Any of the oral pharmaceutical medications described in this application that include alginate, an antacid and an H2RA may be used.

In some embodiments, the oral administration is at night, with the method providing immediate and sustained relief from pain, discomfort and/or symptoms associated with nocturnal episodic heartburn, acid reflux, or both.

Further, the immediate and sustained relief can last longer in duration than that provided by oral treatment with only the antacid, the alginate, or the antacid and the alginate. Also, the immediate and sustained relief provided can be faster than and last at least about as long in duration as that provided with oral treatment with either the histamine H₂-receptor antagonist or the histamine H₂-receptor antagonist and the antacid.

In the context of nocturnal episodic heartburn, from supine reflux, the addition of alginate would not have been expected to provide for more immediate and sustained relief. Alginate is not expected to provide a mechanical block between the stomach and esophagus when a person is lying down, or supine.

The method may be effective to prevent further injury to an esophagus injured by stomach acid from previous episodic heartburn. The method may also be effective to prevent further injury to a larynx or other tissues injured by stomach acid. Regular performance of the method over a period of weeks and months may be effective to promote some degree of healing of the esophagous, larynx and other tissues previously damaged by stomach acid from episodic heartburn.

### EXAMPLES

The following example describes the various aspects and embodiments described above. However, the use of these and other examples anywhere in the specification is illustrative only and in no way limits the scope and meaning of any of the disclosure or of any exemplified term. Likewise, any claimed subject matter is not limited to any particular preferred embodiments described here. Indeed, many modifications and variations may be apparent to those skilled in the art upon reading this specification, and such variations can be made without departing in spirit or in scope from the aspects and embodiments disclosed herein. Any claimed subject matter is therefore to be limited only by the terms of the appended claims along with the full scope of equivalents to which those claims are entitled.

### Example 1

Study to Evaluate the Use of a Famotidine/Antacid/Alginic Acid Combination in Treating Episodic Heartburn

The study was conducted at MetroHealth system, a major teaching hospital of Case Western Reserve University. To be eligible for the trial, adult patients (>18 years of age) were required to report episodic heartburn at least 2 times per week. All patients were provided written informed consent before voluntarily enrolling in the study. Approval was acquired by the Institutional Review Board of The MetroHealth system. Ease of survey use and content were independently verified by a gastroenterologist, internist and registered nurse (4).

Patients with any of the following criteria were excluded: (1) receiving PPIs within the last one month, (2) Helicobacter pylori eradication therapy within the last six months, (3) pregnant or women planning on becoming pregnant at any time during the study, (4) a finding of erosive esophagitis on esophagogastroduodenoscopy (EGD) during the prior three months, (5) a history of erosive esophagitis or Barrett's esophagus, (6) esophageal cancer, or (7) a history of upper gastrointestinal surgery.

Questionnaire

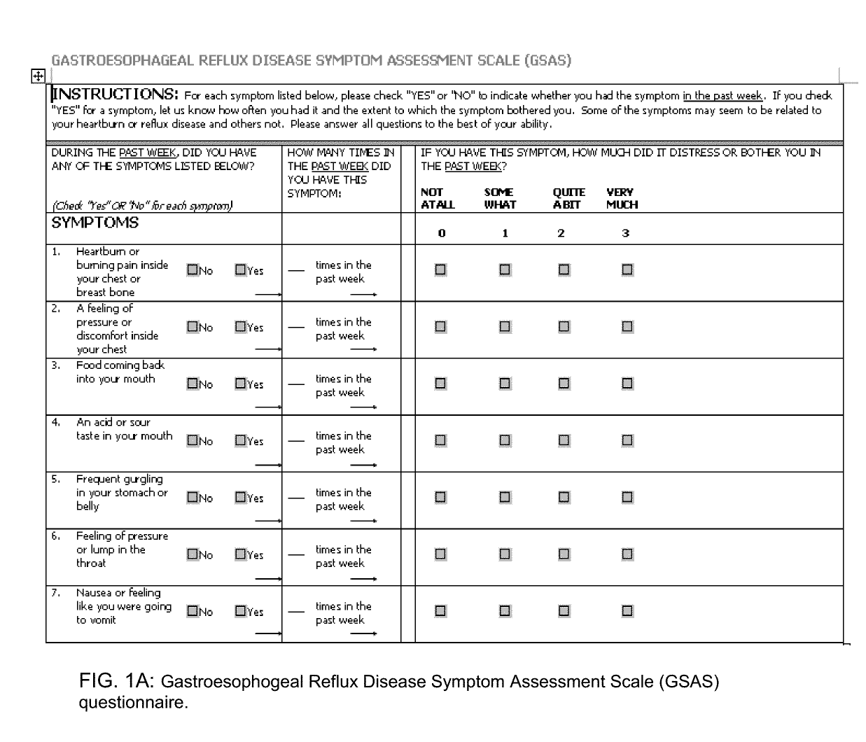

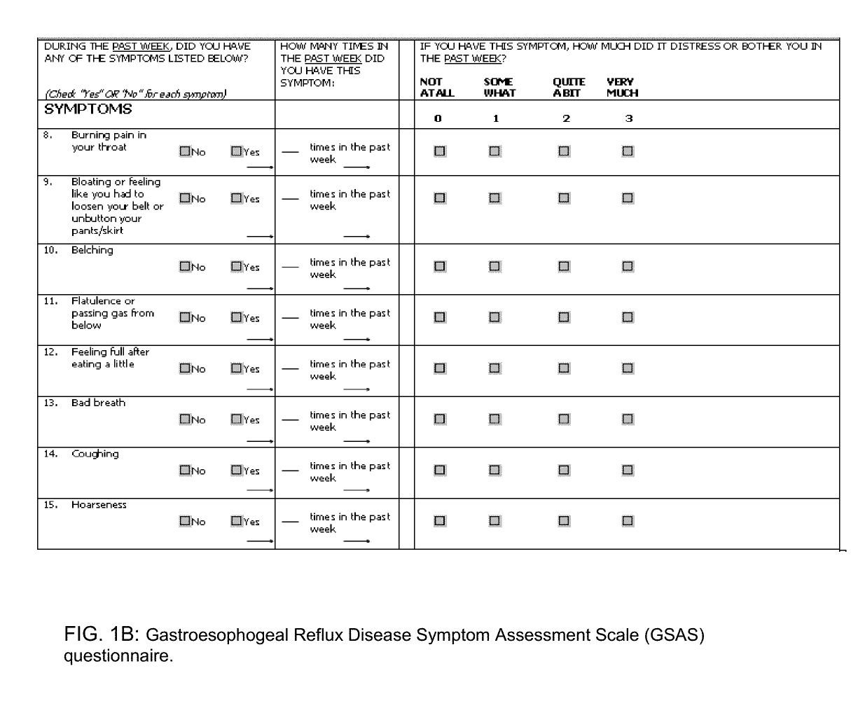

Demographic data were collected that included age, ethnicity, gender, annual household income, smoking status, and alcohol use. GERD symptoms were assessed via a Gastroesophageal Reflux Disease Symptom Assessment Scale (GSAS) questionnaire developed by Johnson & Johnson, an internationally validated a self-administered questionnaire that measures number, severity, and frequency of symptoms, and independently scored based on 15 questions (13, 14). The GSAS questionnaire used in this study is shown in FIGS. 1A and 1B. The GSAS questionnaire is divided into 3 subscales: (A) gastrointestinal distress (gurgling, bloating, belching, flatulence, feeling full), (B) regurgitation and heartburn (heartburn, pressure in chest, food regurgitation, acid/sour taste, lump in the throat, nausea, burning in the throat, bad breath), and (C) upper respiratory manifestations (coughing, hoarseness) (15, 16). Patients first indicated the presence and frequency of each symptom, with severity quantified on a 4-point Likert scale (0=not at all, 1=somewhat, 2=quite a bit and 3=very much). The overall frequency and severity score was calculated as the average of the 15 symptom scores. Any questionnaire with four or more missing symptom scores was considered incomplete (13, 14).

Methods

Seventeen patient volunteers initially met the eligibility criteria and were enrolled in a three-way crossover study where patients were randomized into three groups. Two additional patient volunteers later enrolled in the study, with their results reflected in Example 2. The average age of the initial seventeen patients enrolled was 50.4±3.1 (mean±SE) and the majority of these patients were African American (73%). Their complete demographic details are listed in Table 1.

TABLE 1
Demographic Data Based on Group Randomization

| | Pepcid Complete ® and | | Pepcid Complete ® and | | Sodium Alginate and | |
|---|---|---|---|---|---|---|
| | Sodium Alginate (N = 6) | | Placebo (N = 6) | | Placebo (N = 5) | |
| Age* | 49.2 ± 4.5 | | 58.7 ± 3.2 | | 41.8 ± 7.0 | |
| Gender - no. (%) | | | | | | |
| Male | 3 | (50) | 2 | (33) | 3 | (60) |
| Female | 3 | (50) | 4 | (67) | 2 | (40) |
| Race - no. (%) | | | | | | |
| Caucasian | 3 | (50) | 1 | (17) | 1 | (20) |
| African American | 3 | (50) | 5 | (83) | 3 | (60) |
| Asian | 0 | (0) | 0 | (0) | 0 | (0) |
| Hispanic | 0 | (0) | 0 | (0) | 4 | (80) |
| Other | 0 | (0) | 0 | (0) | 0 | (0) |
| Income - no. (%) | | | | | | |
| <$10,000 | 2 | (33) | 4 | (67) | 4 | (80) |
| $10,000-$30,000 | 2 | (33) | 2 | (33) | 1 | (20) |
| >$30,000 | 2 | (33) | 0 | (0) | 0 | (0) |
| Education - no. (%) | | | | | | |
| Some school | 0 | (0) | 0 | (0) | 0 | (0) |
| High school/GED | 2 | (33) | 1 | (17) | 4 | (80) |
| Some college | 2 | (33) | 5 | (83) | 1 | (20) |
| College or Postgraduate | 2 | (33) | 0 | (0) | 0 | (0) |
| Tobacco Use - no. (%) | | | | | | |
| Yes | 4 | (67) | 2 | (33) | 2 | (40) |
| No | 2 | (33) | 4 | (67) | 3 | (60) |
| Alcohol Use - no. (%) | | | | | | |
| Yes | 3 | (50) | 2 | (33) | 0 | (0) |
| No | 3 | (50) | 4 | (67) | 5 | (100) |

*Mean ± Standard Error.
GED: General Educational Development

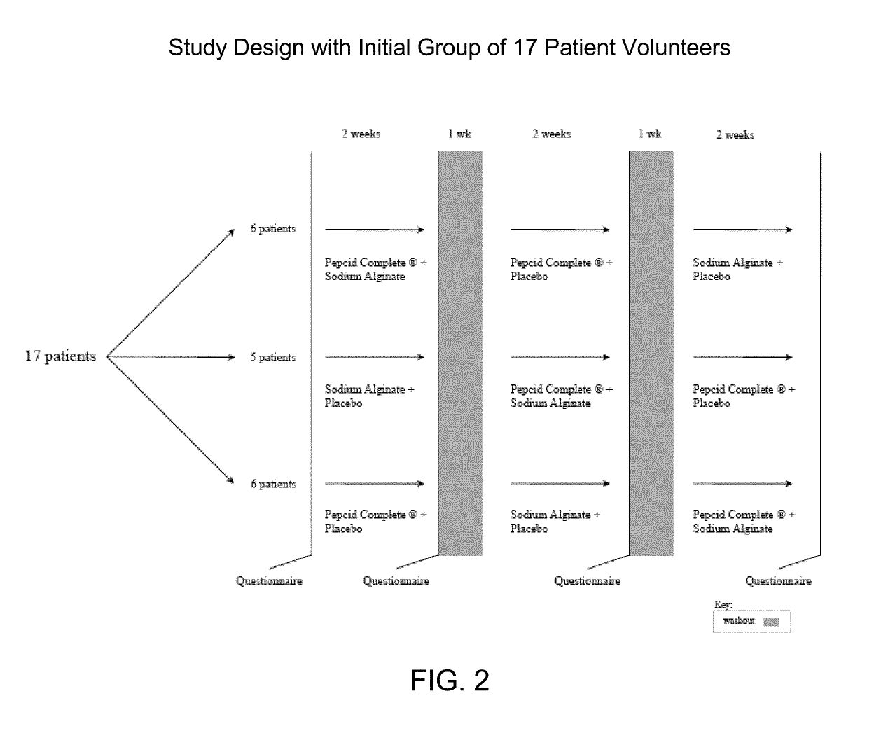

The study design is illustrated in FIG. 2. In the initial two week treatment period, those in the group 1 received Pepcid Complete® (famotidine 10 mg, calcium carbonate 800 mg, magnesium hydroxide 165 mg) and sodium alginate. Those in the group 2 received sodium alginate and placebo. Patients in group 3 received Pepcid Complete® and placebo. In all groups, patients' baseline GERD symptoms were assessed via the GSAS questionnaire. Patients were provided with medications and instructions for on demand dosing in their specific group assignment. At the completion of two weeks of therapy, patients repeated the GSAS questionnaire.

Then all patients underwent a washout period of one week during which no medication was administered. The patients were then crossed over in the following manner: group 1 to group 2, group 2 to group 3, and group 3 to group 1. This sequence was repeated until each group had completed all three combinations, with a total study duration of eight weeks for each patient.

Statistical Analysis

Statistical analysis was then performed on the data, involving a 3-period, 3-treatment crossover design with five patients assigned to each treatment sequence. Each patient received each of the three treatments. Three outcome variables were measured on each patient: symptom score, frequency score and severity score. An overall score and three subgroup scores were obtained for each outcome. Consistent with crossover design analysis, each score was modeled as a mixed effects model with period and treatment as fixed effects and patient nested in sequence as a random effect.

SAS version 9.4 (Statistical Analysis Software, SAS Institute Inc., Cary, N.C., US) was used for statistical analysis. A p-value of less than 0.05 was considered significant after Bonferroni adjustment for multiple comparisons.

Results

Of the seventeen patients enrolled in the study, fifteen patients completed the study. Two patients did not complete the study because one who started in group 3 did not take any of the medications and a second patient who started in group 1 left the study for an unrelated medical issue. Neither provided sufficient data to be included in the analysis. No adverse events were reported by research participants during the study period.

Table 2 shows the results for each of the three groups, expressed as symptom score, frequency score and severity score.

TABLE 2
Mean Symptom, Frequency and Severity Scores (GSAS)

| Symptom Score* | |
|---|---|
| Group 1: Combination (Pepcid Complete ® and Sodium | 5.00 ± 0.60 |
| Alginate) | |
| Group 2: Pepcid Complete ® and Placebo | 6.33 ± 0.60 |
| Group 3: Sodium Alginate and Placebo | 7.47 ± 0.60 |
| Frequency Score* | |
| Group 1: Combination (Pepcid Complete ® and Sodium | 0.86 ± 0.24 |
| Alginate) | |
| Group 2: Pepcid Complete ® and Placebo | 1.34 ± 0.24 |
| Group 3: Sodium Alginate and Placebo | 1.48 ± 0.24 |
| Severity Score* | |
| Group 1: Combination (Pepcid Complete ® and Sodium | 0.37 ± 0.24 |
| Alginate) | |
| Group 2: Pepcid Complete ® and Placebo | 0.58 ± 0.24 |
| Group 3: Sodium Alginate and Placebo | 0.66 ± 0.24 |

*Least Squares (LS) Mean ± Standard Error

From the data in Table 2, there is a surprising decrease in the number, frequency and severity of symptoms when sodium alginate is used in combination with Pepcid Complete®, as compared to either Pepcid Complete® or alginate with placebo. These differences were further analyzed statistically by calculating the LS mean difference and p-values, which are shown in Table 3. Overall, patients receiving the combination of Pepcid Complete® and sodium alginate experienced a greater decrease in number (p-value: <0.0001), frequency (p-value: 0.0010) and severity of symptoms (p-value: <0.0001) compared to patients receiving sodium alginate alone.

TABLE 3
Overall GSAS Scores. Comparing Mean Symptom, Frequency
and Severity Scores for Combination (Pepcid Complete ® and
Sodium Alginate) versus (1) Pepcid Complete ® and
Placebo or (2) Sodium Alginate and Placebo

| | LS Mean Difference* | p-values |
|---|---|---|
| Symptom Score | | |
| Combination vs. Pepcid Complete ® | −1.33 ± 0.48 | 0.0104 |
| Combination vs. Sodium Alginate | −2.47 ± 0.48 | <0.0001 |
| Frequency Score | | |
| Combination vs. Pepcid Complete ® | −0.48 ± 0.17 | 0.0081 |
| Combination vs. Sodium Alginate | −0.62 ± 0.17 | 0.0010 |
| Severity Score | | |
| Combination vs. Pepcid Complete ® | −0.21 ± 0.05 | 0.0006 |
| Combination vs. Sodium Alginate | −0.29 ± 0.05 | <0.0001 |

*Least Squares (LS) Mean Difference ± Standard Error (SE)

Further analysis was performed by focusing on regurgitation and the heartburn predominant symptoms. Subgroup analysis demonstrated a greater relief of regurgitation and heartburn predominant symptom number, frequency and severity in patients receiving combination therapy compared to Pepcid Complete® or sodium alginate alone. See Table 4.

TABLE 4
Subgroup Analysis for Regurgitation and Heartburn. Comparing
Mean Symptom, Frequency and Severity Scores for Combination
(Pepcid Complete ® and Sodium Alginate) versus (1)
Pepcid Complete ® and Placebo OR (2) Sodium Alginate and Placebo.

| | LS Mean Difference* | p-values |
|---|---|---|
| Symptom Score | | |
| Combination vs. Pepcid Complete ® | −1.47 ± 0.40 | 0.0011 |
| Combination vs. Sodium Alginate | −1.40 ± 0.40 | 0.0017 |
| Frequency Score | | |
| Combination vs. Pepcid Complete ® | −0.82 ± 0.17 | <0.0001 |
| Combination vs. Sodium Alginate | −0.63 ± 0.17 | 0.0013 |
| Severity Score | | |
| Combination vs. Pepcid Complete ® | −0.37 ± 0.09 | 0.0004 |
| Combination vs. Sodium Alginate | −0.33 ± 0.09 | 0.0011 |

*Least Squares (LS) Mean Difference ± Standard Error (SE)

The combination of Pepcid Complete®, antacid and sodium alginate was superior to sodium alginate in providing relief from gastrointestinal distress (Table 5). However, no significant differences were observed among the three groups for upper respiratory manifestations, as shown in Table 6.

TABLE 5
Subgroup Analysis for Gastrointestinal Distress. Comparing
Mean Symptom, Frequency and Severity Scores for Combination
(Pepcid Complete ® and Sodium Alginate) versus (1)
Pepcid Complete ® and Placebo OR (2) Sodium Alginate and Placebo.

| | LS Mean Difference* | p-values |
|---|---|---|
| Symptom Score | | |
| Combination vs. Pepcid Complete ® | 0.20 ± 0.30 | 0.5161 |
| Combination vs. Sodium Alginate | −0.80 ± 0.30 | 0.0141 |
| Frequency Score | | |
| Combination vs. Pepcid Complete ® | −0.09 ± 0.36 | 0.7963 |
| Combination vs. Sodium Alginate | −0.77 ± 0.36 | 0.0401 |
| Severity Score | | |
| Combination vs. Pepcid Complete ® | −0.04 ± 0.09 | 0.6764 |
| Combination vs. Sodium Alginate | −0.29 ± 0.09 | 0.0047 |

*Least Squares (LS) Mean Difference ± Standard Error (SE)

TABLE 6
Subgroup Analysis for Upper Respiratory Manifestations. Comparing
Mean Symptom, Frequency and Severity Scores for Combination
(Pepcid Complete ® and Sodium Alginate) versus (1)
Pepcid Complete ® and Placebo OR (2) Sodium Alginate and Placebo.

| | LS Mean Difference* | p-values |
|---|---|---|
| Symptom Score | | |
| Combination vs. Pepcid Complete ® | −0.07 ± 0.18 | 0.7168 |
| Combination vs. Sodium Alginate | −0.27 ± 0.18 | 0.1544 |
| Frequency Score | | |
| Combination vs. Pepcid Complete ® | −0.20 ± 0.17 | 0.2421 |
| Combination vs. Sodium Alginate | −0.33 ± 0.17 | 0.0566 |
| Severity Score | | |
| Combination vs. Pepcid Complete ® | −0.07 ± 0.12 | 0.5835 |
| Combination vs. Sodium Alginate | −0.20 ± 0.12 | 0.1078 |

*Least Squares (LS) Mean Difference ± Standard Error (SE)

### Example 2

Study to Evaluate the Use of a Famotidine/Antacid/Alginic Acid Combination in Treating Episodic Heartburn

The study described in Example 1 enrolled two additional patients who met the eligibility criteria. The added patients were adults (>18 years of age) and fulfilled the requirement to report episodic heartburn at least 2 times per week. The added patients were provided written informed consent before voluntarily enrolling in the study.

Methods

The nineteen patient volunteers were randomized into three groups as described in Example 1. The average age of patients enrolled was 51.3±2.9 (mean±SE) and the majority of patients were African American (68%). Complete demographic details are listed in Table 7.

TABLE 7
Demographic Data Based on Group Randomization

| Age* | 51.3 ± 2.9 | |
|---|---|---|
| Gender - no. (%) | | |
| Male | 9 | (47.4) |
| Female | 10 | (52.6) |
| Race - no. (%) | | |
| Caucasian | 5 | (26.3) |
| African American | 13 | (68.4) |
| Asian | 0 | (0) |
| Hispanic | 1 | (5.3) |
| Other | 0 | (0) |
| Income - no. (%) | | |
| <$10,000 | 12 | (63.2) |
| $10,000-$30,000 | 5 | (26.3) |
| >$30,000 | 2 | (10.5) |
| Education - no. (%) | | |
| Some school | 1 | (5.3) |
| High school/GED | 8 | (42.1) |
| Some college | 8 | (42.1) |
| College or Postgraduate | 2 | (10.5) |
| Tobacco Use - no. (%) | | |
| Yes | 10 | (52.6) |
| No | 9 | (47.4) |
| Alcohol Use - no. (%) | | |
| Yes | 6 | (31.6) |
| No | 13 | (68.4) |

*Mean ± Standard Error.
GED: General Educational Development

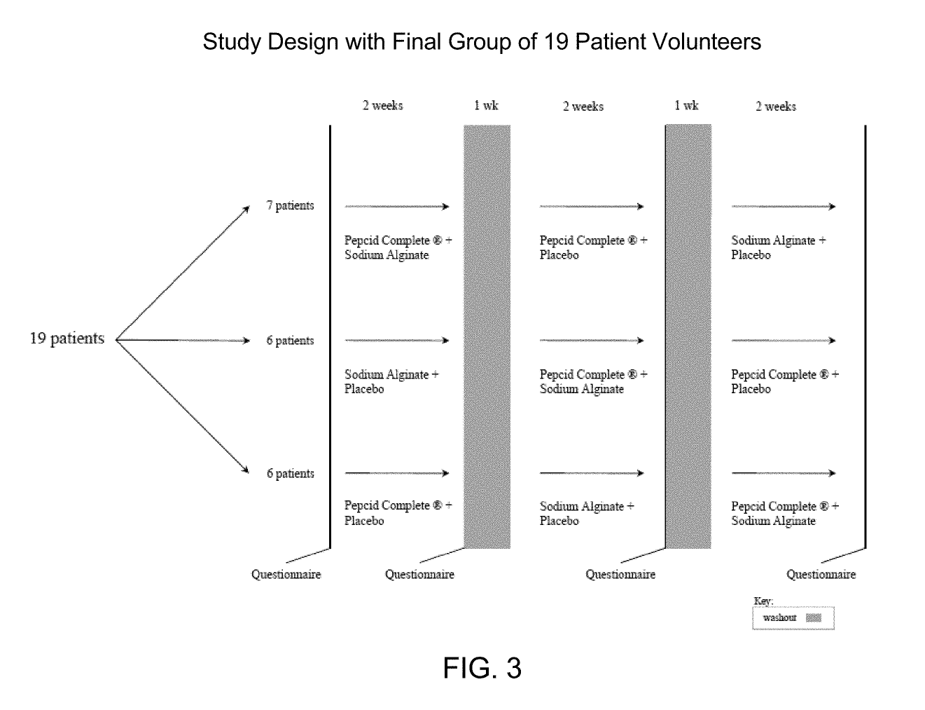

The study design is illustrated in FIG. 3. In the initial two week treatment period, those in the group 1 received Pepcid Complete® (famotidine 10 mg, calcium carbonate 800 mg, magnesium hydroxide 165 mg) and sodium alginate. Those in the group 2 received sodium alginate and placebo. Patients in group 3 received Pepcid Complete® and placebo. In all groups, patients' baseline GERD symptoms were assessed via the GSAS questionnaire. Patients were provided with medications and instructions for on demand dosing in their specific group assignment. At the completion of two weeks of therapy, patients repeated the GSAS questionnaire.

Then all patients underwent a washout period of one week during which no medication was administered. The patients were then crossed over in the following manner: group 1 to group 2, group 2 to group 3, and group 3 to group 1. This sequence was repeated until each group had completed all three combinations, with a total study duration of eight weeks for each patient.

Statistical Analysis

Statistical analysis was then performed on the data, involving a 3-period, 3-treatment crossover design with five patients assigned to each treatment sequence. Each patient received each of the three treatments. Three outcome variables were measured on each patient: symptom score, frequency score and severity score. An overall score and three subgroup scores were obtained for each outcome. Consistent with crossover design analysis, each score was modeled as a mixed effects model with period and treatment as fixed effects and patient nested in sequence as a random effect.

SAS version 9.4 (Statistical Analysis Software, SAS Institute Inc., Cary, N.C., US) was used for statistical analysis. A p-value of less than 0.05 was considered significant after Bonferroni adjustment for multiple comparisons.

Results

Of the nineteen patients enrolled in the study, sixteen patients completed the study. Three patients did not complete the study because one who started in group 3 did not take any of the medications and the other two patients who started in group 1 left the study for unrelated medical issues. None provided sufficient data to be included in the analysis. No adverse events were reported by research participants during the study period.

Table 8 shows the results for each of the three groups, expressed as symptom score, frequency score and severity score.

TABLE 8
Mean Symptom, Frequency and Severity Scores (GSAS)

| Symptom Score* | |
|---|---|
| Group 1: Combination (Pepcid Complete ® and Sodium | 5.28 ± 0.60 |
| Alginate) | |
| Group 2: Pepcid Complete ® and Placebo | 6.61 ± 0.62 |
| Group 3: Sodium Alginate and Placebo | 7.74 ± 0.62 |
| Frequency Score* | |
| Group 1: Combination (Pepcid Complete ® and Sodium | 0.99 ± 0.24 |
| Alginate) | |
| Group 2: Pepcid Complete ® and Placebo | 1.40 ± 0.25 |
| Group 3: Sodium Alginate and Placebo | 1.57 ± 0.25 |
| Severity Score* | |
| Group 1: Combination (Pepcid Complete ® and Sodium | 0.46 ± 0.10 |
| Alginate) | |
| Group 2: Pepcid Complete ® and Placebo | 0.66 ± 0.11 |
| Group 3: Sodium Alginate and Placebo | 0.74 ± 0.11 |

*Least Squares (LS) Mean ± Standard Error

From the data in Table 8, there is a surprising decrease in the number, frequency and severity of symptoms when sodium alginate is used in combination with Pepcid Complete®, as compared to either Pepcid Complete® or alginate with placebo. These differences were further analyzed statistically by calculating the LS mean difference and p-values, which are shown in Table 9. Overall, patients receiving the combination of Pepcid Complete® and sodium alginate experienced a greater decrease in number (p-value: <0.001), frequency (p-value: 0.001) and severity of symptoms (p-value: <0.001) compared to patients receiving sodium alginate alone.

TABLE 9
Overall GSAS Scores. Comparing Mean Symptom, Frequency
and Severity Scores for Combination (Pepcid Complete ® and
Sodium Alginate) versus (1) Pepcid Complete ® and
Placebo or (2) Sodium Alginate and Placebo

| | LS Mean Difference* | p-values |
|---|---|---|
| Symptom Score | | |
| Combination vs. Pepcid Complete ® | −1.33 ± 0.45 | 0.007 |
| Combination vs. Sodium Alginate | −2.46 ± 0.45 | <0.001 |
| Frequency Score | | |
| Combination vs. Pepcid Complete ® | −0.40 ± 0.16 | 0.016 |
| Combination vs. Sodium Alginate | −0.58 ± 0.16 | 0.001 |
| Severity Score | | |
| Combination vs. Pepcid Complete ® | −0.20 ± 0.05 | 0.001 |
| Combination vs. Sodium Alginate | −0.28 ± 0.05 | <0.001 |

*Least Squares (LS) Mean Difference ± Standard Error (SE)

Further analysis was performed by focusing on regurgitation and heartburn predominant symptoms. Subgroup analysis demonstrated a greater relief of regurgitation and heartburn predominant symptom number, frequency and severity in patients receiving combination therapy compared to Pepcid Complete® or sodium alginate alone. See Table 10.

TABLE 10
Subgroup Analysis for Regurgitation and Heartburn. Comparing
Mean Symptom, Frequency and Severity Scores for Combination
(Pepcid Complete ® and Sodium Alginate) versus (1)
Pepcid Complete ® and Placebo OR (2) Sodium Alginate and Placebo.

| | LS Mean Difference* | p-values |
|---|---|---|
| Symptom Score | | |
| Combination vs. Pepcid Complete ® | −1.53 ± 0.38 | <0.001 |
| Combination vs. Sodium Alginate | −1.43 ± 0.38 | <0.001 |
| Frequency Score | | |
| Combination vs. Pepcid Complete ® | −0.79 ± 0.16 | <0.001 |
| Combination vs. Sodium Alginate | −0.61 ± 0.16 | <0.001 |
| Severity Score | | |
| Combination vs. Pepcid Complete ® | −0.34 ± 0.09 | <0.001 |
| Combination vs. Sodium Alginate | −0.30 ± 0.09 | 0.003 |

*Least Squares (LS) Mean Difference ± Standard Error (SE)

No significant differences were observed among the three groups for gastrointestinal distress (Table 11) or upper respiratory manifestations, as shown in Table 12.

TABLE 11
Subgroup Analysis for Gastrointestinal Distress. Comparing
Mean Symptom, Frequency and Severity Scores for Combination
(Pepcid Complete ® and Sodium Alginate) versus (1)
Pepcid Complete ® and Placebo OR (2) Sodium Alginate and Placebo.

| | LS Mean Difference* | p-values |
|---|---|---|
| Symptom Score | | |
| Combination vs. Pepcid Complete ® | 0.28 ± 0.29 | 0.330 |
| Combination vs. Sodium Alginate | −0.75 ± 0.29 | 0.014 |
| Frequency Score | | |
| Combination vs. Pepcid Complete ® | 0.03 ± 0.34 | 0.935 |
| Combination vs. Sodium Alginate | −0.65 ± 0.34 | 0.064 |
| Severity Score | | |
| Combination vs. Pepcid Complete ® | −0.01 ± 0.09 | 0.892 |
| Combination vs. Sodium Alginate | −0.27 ± 0.09 | 0.006 |

*Least Squares (LS) Mean Difference ± Standard Error (SE)

TABLE 12
Subgroup Analysis for Upper Respiratory Manifestations. Comparing
Mean Symptom, Frequency and Severity Scores for Combination
(Pepcid Complete ® and Sodium Alginate) versus (1)
Pepcid Complete ® and Placebo OR (2) Sodium Alginate and Placebo.

| | LS Mean Difference* | p-values |
|---|---|---|
| Symptom Score | | |
| Combination vs. Pepcid Complete ® | −0.11 ± 0.16 | 0.497 |
| Combination vs. Sodium Alginate | −0.36 ± 0.16 | 0.028 |
| Frequency Score | | |
| Combination vs. Pepcid Complete ® | −0.17 ± 0.16 | 0.284 |
| Combination vs. Sodium Alginate | −0.32 ± 0.16 | 0.051 |
| Severity Score | | |
| Combination vs. Pepcid Complete ® | −0.07 ± 0.11 | 0.542 |
| Combination vs. Sodium Alginate | −0.22 ± 0.11 | 0.053 |

*Least Squares (LS) Mean Difference ± Standard Error (SE)

The claimed subject matter is not to be limited in scope by the specific embodiments described herein. Indeed, various modifications of the claimed subject matter in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

1. Wolfe M M, Sachs G. Acid suppression: optimizing therapy for gastroduodenal ulcer healing, gastroesophageal reflux disease, and stress-related erosive syndrome. Gastroenterology. 2000; 118(2 Suppl 1):S9-31. Epub 2000 Jun. 27. PubMed PMID: 10868896.
2. Lowe R C, Wolfe M M. The pharmacological management of gastroesophageal reflux disease. Minerva gastroenterologica e dietologica. 2004; 50(3):227-37. Epub 2005 Feb. 25. PubMed PMID: 15729198.
3. Barrison A F, Jarboe L A, Weinberg B M, Nimmagadda K, Sullivan L M, Wolfe M M. Patterns of proton pump inhibitor use in clinical practice. The American journal of medicine. 2001; 111(6):469-73. Epub 2001 Nov. 3. PubMed PMID: 11690573.
4. Sheikh I, Waghray A, Waghray N, Dong C, Wolfe M M. Consumer use of over-the-counter proton pump inhibitors in patients with gastroesophageal reflux disease. The American journal of gastroenterology. 2014; 109(6):789-94. Epub 2014 Jun. 5. doi: 10.1038/ajg.2013.421. PubMed PMID: 24896751.
5. Wolfe M M N T. The Fire Inside: W.W. Norton and Company; 1996 1996.
6. Sontag S J. The medical management of reflux esophagitis. Role of antacids and acid inhibition. Gastroenterology clinics of North America. 1990; 19(3):683-712. Epub 1990 Sep. 1. PubMed PMID: 1977703.
7. Rohof W O, Bennink R J, Smout A J, Thomas E, Boeckxstaens G E. An alginate-antacid formulation localizes to the acid pocket to reduce acid reflux in patients with gastroesophageal reflux disease. Clinical gastroenterology and hepatology: the official clinical practice journal of the American Gastroenterological Association. 2013; 11(12):1585-91; quiz e90. Epub 2013 May 15. doi: 10.1016/j.cgh.2013.04.046. PubMed PMID: 23669304.
8. Tytgat G N, Nio C Y. The medical therapy of reflux oesophagitis. Bailliere's clinical gastroenterology. 1987; 1(4):791-807. Epub 1987 Oct. 1. PubMed PMID: 2896523.
9. Cloud M L, Offen W W, Robinson M. Nizatidine versus placebo in gastroesophageal reflux disease: a 12-week, multicenter, randomized, double-blind study. The American journal of gastroenterology. 1991; 86(12):1735-42. Epub 1991 Dec. 1. PubMed PMID: 1962618.
10. Colin-Jones D G. The role and limitations of H2-receptor antagonists in the treatment of gastro-oesophageal reflux disease. Alimentary pharmacology & therapeutics. 1995; 9 Suppl 1:9-14. Epub 1995 Jan. 1. PubMed PMID: 7495945.
11. Steinberg W M, Lewis J H, Katz D M. Antacids inhibit absorption of cimetidine. The New England journal of medicine. 1982; 307(7):400-4. Epub 1982 Aug. 12. doi: 10.1056/nejm198208123070703. PubMed PMID: 7088113.
12. Feldman M. Comparison of the effects of over-the-counter famotidine and calcium carbonate antacid on postprandial gastric acid. A randomized controlled trial. Jama. 1996; 275(18):1428-31. Epub 1996 May 8. PubMed PMID: 8618369.
13. Rothman M, Farup C, Stewart W, Helbers L, Zeldis J. Symptoms associated with gastroesophageal reflux disease: development of a questionnaire for use in clinical trials. Digestive diseases and sciences. 2001; 46(7):1540-9. Epub 2001 Aug. 2. PubMed PMID: 11478508.
14. Damiano A, Handley K, Adler E, Siddique R, Bhattacharyja A. Measuring symptom distress and health-related quality of life in clinical trials of gastroesophageal reflux disease treatment: further validation of the Gastroesophageal Reflux Disease Symptom Assessment Scale (GSAS). Digestive diseases and sciences. 2002; 47(7):1530-7. Epub 2002 Jul. 27. PubMed PMID: 12141813.
15. Damiano A, Siddique R, Xu X, Johanson J, Sloan S. Reductions in symptom distress reported by patients with moderately severe, nonerosive gastroesophageal reflux disease treated with rabeprazole. Digestive diseases and sciences. 2003; 48(4):657-62. Epub 2003 May 14. PubMed PMID: 12741452.
16. Jha L K, Fass R, Gadam R, Maradey-Romero C, Nasrollah L, Hershcovici T, Quan S F, Dickman R. The Effect of Ramelteon on Heartburn Symptoms of Patients With Gastroesophageal Reflux Disease and Chronic Insomnia: A Pilot Study. Journal of clinical gastroenterology. 2015. Epub 2015 Apr. 19. doi: 10.1097/mcg.0000000000000322. PubMed PMID: 25887111.

1. An oral pharmaceutical medication for providing immediate and sustained relief from pain, discomfort and/or symptoms associated with acid reflux and/or episodic heartburn in a human, said oral pharmaceutical medication comprising:
an antacid in an amount effective to substantially neutralize gastric acid;
alginate; and
a histamine H₂-receptor antagonist in an amount effective to substantially inhibit or block gastric acid secretion.

2. The oral pharmaceutical medication of claim 1, wherein said oral pharmaceutical medication is effective to provide immediate and sustained relief from pain, discomfort and/or symptoms associated with episodic heartburn in the human.

3. The oral pharmaceutical medication of claim 1, wherein said oral pharmaceutical medication is effective to provide immediate and sustained relief from pain, discomfort and/or symptoms associated with acid reflux in the human.

4. (canceled)

5. The oral pharmaceutical medication of claim 1, wherein said immediate and sustained relief lasts longer in duration than when the human is orally treated with either the antacid, the alginate, or the antacid and the alginate.

6. The oral pharmaceutical medication of claim 1, wherein said immediate sustained relief is faster than and lasts at least about as long in duration as when the human is orally treated with either the histamine H₂-receptor antagonist or the histamine H₂-receptor antagonist and the antacid.

7. The oral pharmaceutical medication of claim 1, wherein said oral pharmaceutical medication is in a liquid dosage form or a solid dosage form.

8. (canceled)

9. The oral pharmaceutical medication of claim 1, wherein said histamine H₂-receptor antagonist is famotidine.

10. The oral pharmaceutical medication of claim 9, wherein said famotidine is present in an amount of between 2 to 30 mg.

11. The oral pharmaceutical medication of claim 1, wherein said antacid is present in an amount between 200 to 1500 mg and said alginate is present in an amount between 100 to 1500 mg.

12. (canceled)

13. The oral pharmaceutical medication of claim 1, wherein said histamine H₂-receptor antagonist is cimetidine or famotidine.

14. The oral pharmaceutical medication of claim 13, wherein said cimetidine is present in an amount of between about 50 mg and about 400 mg, or wherein said ranitidine is present in an amount of between about 25 mg and about 300 mg.

15. (canceled)

16. (canceled)

17. The oral pharmaceutical medication of claim 1, wherein said histamine H₂-receptor antagonist is selected from the group consisting of famotidine and nizatidine.

18. The oral pharmaceutical medication of claim 1, wherein said antacid is selected from the group consisting of aluminum hydroxide, calcium carbonate, magnesium hydroxide, sodium bicarbonate and mixtures thereof, wherein optionally said antacid is a flavored antacid, optionally said antacid is a high potency antacid, optionally said oral medication further includes an effective amount of an antiflatulent, and optionally said antiflatulent is simethicone.

19.-22. (canceled)

23. The oral pharmaceutical medication of claim 1, wherein said antacid is aluminum hydroxide and magnesium hydroxide, and wherein said histamine H₂-receptor antagonist is cimetidine or ranitidine.

24. (canceled)

25. A method of providing immediate and sustained relief from pain, discomfort and/or symptoms associated with acid reflux and/or episodic heartburn in a human, said method comprising:
orally administering to a human, together or substantially together, an antacid in an amount effective to substantially neutralize gastric acid, alginate, and a histamine H₂-receptor antagonist in an amount effective to substantially inhibit or block gastric add secretion for providing the human with immediate and sustained relief from pain, discomfort and or symptoms associated with acid reflux and/or episodic heartburn.

26. The method of claim 25, wherein the immediate and sustained relief provided lasts longer in duration than when the human is orally treated with either the antacid, the alginate, or a combination of the antacid and the alginate.

27. The method of claim 25, wherein the immediate and sustained relief provided is fluster than and lasts at least about as long in duration as when the human is orally treated with either the histamine H₂-receptor antagonist or a combination of the histamine H₂-receptor antagonist and the antacid.

28. The method of claim 25, wherein the histamine H₂-receptor antagonist is cimetidine.

29. The method of claim 28, the cimetidine is administered in a daily dosage amount of between about 100 mg and about 1600 mg or the ranitidine is administered in a daily dosage amount of between about 50 mg and about 450 mg.

30. (canceled)

31. (canceled)

32. The method of claim 25, wherein the histamine H₂-receptor antagonist is selected from the group consisting of famotidine and nizatidine, and wherein optionally the nizatidine is administered in a daily dosage amount of between about 50 and about 450 mg, and wherein optionally the famotidine is administered in a daily dosage amount of between about 5 mg to about 40 mg.

33. The method of claim 25, wherein the antacid is selected from the group consisting of aluminum hydroxide, calcium carbonate, magnesium hydroxide, sodium bicarbonate and mixtures thereof, wherein optionally said antacid is a flavored antacid, optionally said antacid is a high potency antacid, optionally said oral medication further includes an effective amount of an antiflatulent, and optionally said antiflatulent is simethicone.

34.-37. (canceled)

38. The method of claim 25, wherein the antacid and the histamine H₂-receptor antagonist are administered in a dosage form selected from a group consisting of liquid and solid dosage forms and mixtures thereof.
